(19) United States
(12) Patent Application Publication
(10) Pub. No.: US 2022/0040400 A1
(43) Pub. Date: Feb. 10, 2022

(54) Differential Collapse Wound Dressings

(71) Applicant: KCI Licensing, Inc., San Antonio, TX (US)

(72) Inventors: Timothy Mark ROBINSON, Shillingstone (GB); Christopher Brian LOCKE, Bournemouth (GB)

(21) Appl. No.: 17/275,245

(22) PCT Filed: Sep. 11, 2019
(86) PCT No.: PCT/US2019/050633

§ 371 Date: Mar. 11, 2021

Related U.S. Application Data
(60) Provisional application No. 62/731,512, filed on Sep. 14, 2018.

(51) Int. Cl. A61M 1/00; A61F 13/02; A61F 13/00

(52) U.S. Cl. CPC A61M 1/915; A61F 13/0216; A61F 2013/00617; A61F 13/0289; A61F 13/00063

(57) ABSTRACT

Dressings and kits for use in negative-pressure therapy are provided herein comprising one or more manifolds and a polymer film laminated to the one or more manifolds. At least one manifold is felted and the manifolds may be placed in a stacked configuration and differentially collapse under negative pressure. Methods of making and using the dressings are also provided herein.

### RELATED APPLICATIONS

The present application claims priority to U.S. Provisional Patent Application No. 62/731,512, entitled Differential Collapse Wound Dressings,” filed Sep. 14, 2018, which is incorporated herein by reference for all purposes.

### TECHNICAL FIELD

The invention set forth in the appended claims relates generally to tissue treatment systems and more particularly, but without limitation, to differential collapse wound dressings.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” “sub-atmospheric pressure” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for reducing tissue ingrowth and increasing granulation in a negative-pressure therapy environment are set forth in the appended claims. Illustrative embodiments are also provided to enable a person skilled in the art to make and use the claimed subject matter.

For example, in some embodiments, dressings are provided which are configured to variably collapse under negative pressure.

More generally, dressings are provided for use with negative-pressure therapy comprising one or more manifolds and a fenestrated polymer film coupled to the one or more manifolds. One or more manifolds present in the dressing are felted and are configured to differentially collapse during negative pressure wound therapy.

In some example embodiments, one, two or three felted manifolds are present in the dressing, optionally in combination with non-felted manifolds, having different degrees of firmness and are configured to be in a stacked configuration with manifolds having lower firmness values on a wound bottom or bed side of a wound, and manifolds having higher firmness values on a wound opening side of a wound.

In some example embodiments, a manifold comprises a polymer foam, such as a polyurethane foam or a polyethylene foam.

Alternatively, other example embodiments include methods of making a dressing described herein comprising felting at least one manifold to a desired degree of firmness and laminating a polymer film to the manifold.

In some example embodiments, the polymer film is fenestrated before or after lamination, or in a one-step process along with lamination.

Alternatively, other example embodiments include methods of treating a tissue site, such as a surface wound, with negative pressure comprising applying a dressing described herein to the tissue site; sealing the dressing to epidermis adjacent to the tissue site; fluidly coupling the dressing to a negative-pressure source; and applying negative pressure from the negative-pressure source to the dressing and promoting healing and tissue granulation.

Alternatively, other example embodiments include wound therapy kits. The wound therapy kits described herein may comprise two or more manifolds having different firmness values, optionally having a fenestrated polymer film laminated thereon. At least one of the manifolds is a felted manifold. The kits may further comprises one or more drapes, and one or more dressing interfaces.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a graph illustrating additional details of example pressure control modes that may be associated with some embodiments of the therapy system of FIG. 1;

FIG. 3 is a graph illustrating additional details that may be associated with another example pressure control mode in some embodiments of the therapy system of FIG. 1;

FIG. 4 is a chart illustrating details that may be associated with an example method of operating the therapy system of FIG. 1;

FIG. 5 is a schematic diagram illustrating additional details of an example of a tissue interface that may be associated with some embodiments of the therapy system of FIG. 1; and

FIG. 6 is a schematic diagram illustrating additional details that may be associated with some embodiments of a manifold.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

### Therapy System

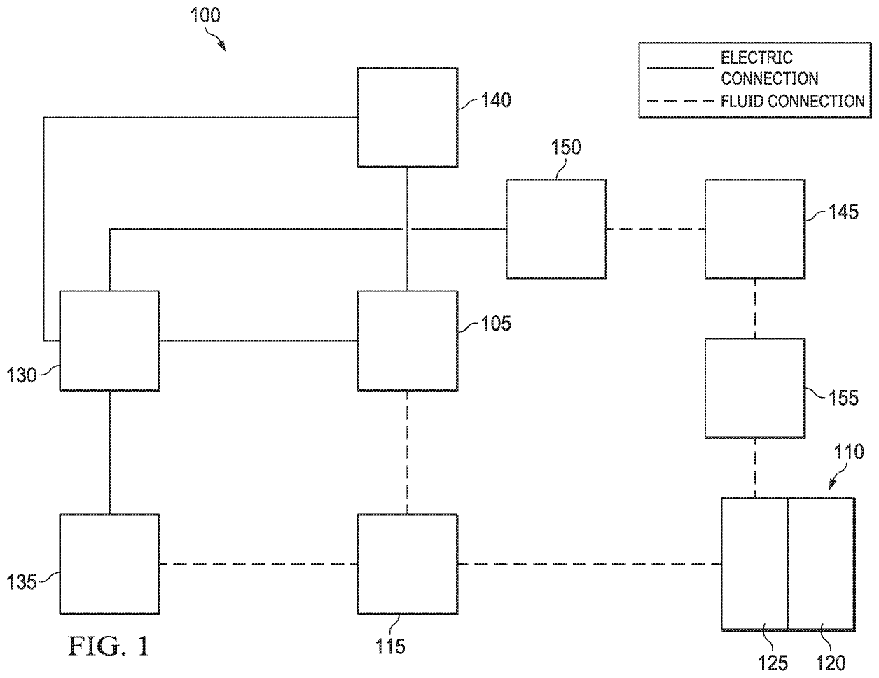

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, full or partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical (wired or wireless), or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, absorbent, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

### Tissue Interface

As noted above, the dressing 110 may comprise or consist essentially of a tissue interface 120, a cover 125, or both in some embodiments. The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of one or more manifolds. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, a manifold may comprise or consist essentially of reticulated foam having pore sizes and free volume that may vary according to needs of a prescribed therapy. For example, reticulated foam having a free volume of at least 40%, at least 50%, at least 60%, at least 70%, at least 80%, or at least 90%, may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of a manifold may be at least 10 pounds per square inch. A manifold may have a tear strength of at least 2.5 pounds per inch. In some embodiments, a manifold may be foam comprised of polyols such as polyester or polyether, isocyanate such as toluene diisocyanate, and polymerization modifiers such as amines and tin compounds. In some examples, a manifold may be reticulated polyurethane foam such as found in GRANUFOAM™ dressing or V.A.C. VERAFLO™ dressing, both available from Kinetic Concepts, Inc. of San Antonio, Tex.

Other suitable materials for the one or more manifold may include non-woven fabrics (Libeltex, Freudenberg), three-dimensional (3D) polymeric structures (molded polymers, embossed and formed films, and fusion bonded films [Supracore]), and mesh, for example.

In some examples, a manifold may include a 3D textile, such as various textiles commercially available from Baltex, Muller, and Heathcoates. A 3D textile of polyester fibers may be particularly advantageous for some embodiments. For example, a manifold may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 mm may be particularly advantageous for some embodiments. Such a puncture-resistant fabric may have a warp tensile strength of about 330-340 kilograms and a weft tensile strength of about 270-280 kilograms in some embodiments. Another particularly suitable material may be a polyester spacer fabric having a weight of about 470 grams per square meter, which may have a thickness of about 4-5 mm in some embodiments. Such a spacer fabric may have a compression strength of about 20-25 kilopascals (at 40% compression). Additionally or alternatively, a manifold may comprise or consist of a material having substantial linear stretch properties, such as a polyester spacer fabric having 2-way stretch and a weight of about 380 grams per square meter. A suitable spacer fabric may have a thickness of about 3-4 mm, and may have a warp and weft tensile strength of about 30-40 kilograms in some embodiments. The fabric may have a close-woven layer of polyester on one or more opposing faces in some examples. In some embodiments, a woven layer may be advantageously disposed on a manifold to face a tissue site.

The thickness of a manifold may also vary according to needs of a prescribed therapy. For example, the thickness of a manifold may be decreased to reduce tension on peripheral tissue. The thickness of a manifold can also affect the conformability of the tissue interface 120. In some embodiments, a manifold thickness, e.g. for a suitable foam, may be in a range of about 3 mm to 10 mm, preferably about 6 mm to about 8 mm. Fabrics, including suitable 3D textiles and spacer fabrics, may have a thickness in a range of about 2 mm to about 8 mm.

A manifold disclosed herein may be either hydrophobic or hydrophilic. In an example in which a manifold may be hydrophilic, the manifold may also wick fluid away from a tissue site, while continuing to distribute negative pressure to the tissue site. The wicking properties of a manifold may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, a manifold may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. A manifold may further serve as a scaffold for new cell-growth, or a scaffold material may be used in conjunction with a manifold to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. Additional embodiments of manifolds for use in a dressing 110 are discussed further herein.

In addition to the tissue interface 120, the dressing 110 may further include the cover 125. In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a non-porous polymer drape or film, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S. A., Colombes, France; and Inspire 2301 and Inpsire 2327 polyurethane films, commercially available from Coveris Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

### NPWT

The dressings disclosed herein may be used with negative-pressure therapy. In some embodiments, the dressing 110 disclosed herein may be used for at least 5, 6, 7, 8, 9, 10, 11 or 12 days to promote granulation and/or minimize tissue in-growth with a source of negative pressure. For example, the dressing 110 disclosed herein may remain on a tissue site, such as a surface wound, for at least 5 to 7 days.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

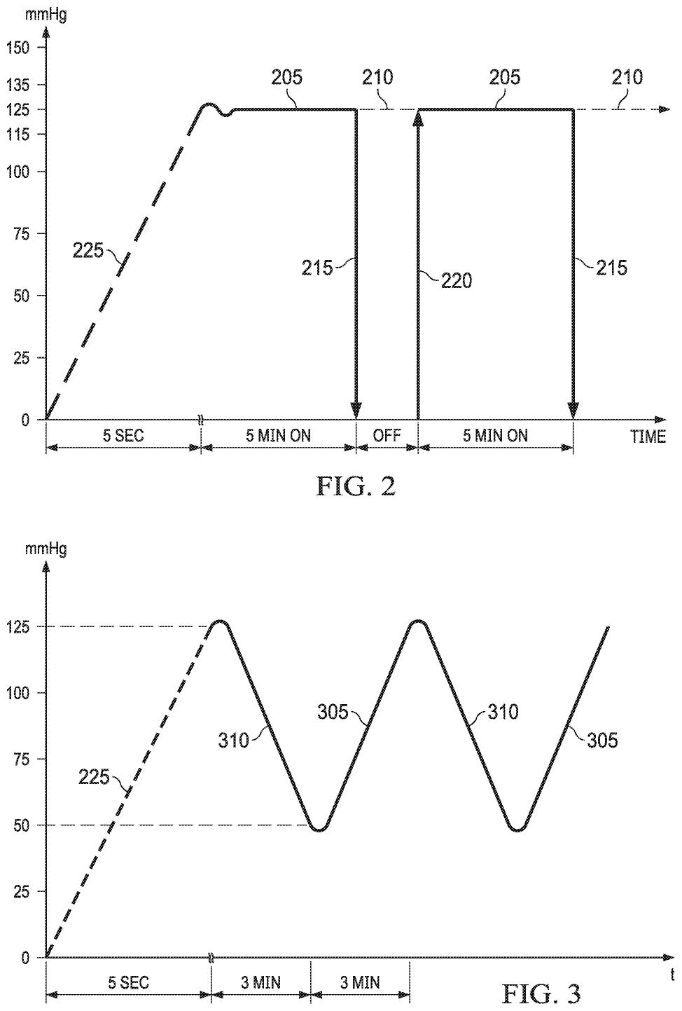

FIG. 2 is a graph illustrating additional details of an example control mode that may be associated with some embodiments of the controller 130. In some embodiments, the controller 130 may have a continuous pressure mode, in which the negative-pressure source 105 is operated to provide a constant target negative pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time and the y-axis represents negative pressure generated by the negative-pressure source 105 over time. In the example of FIG. 2, the controller 130 can operate the negative-pressure source 105 to cycle between a target pressure and atmospheric pressure. For example, the target pressure may be set at a value of 125 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time, as indicated by the solid line 220, may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a negative pressure of 50 and 125 mmHg with a rise time 305 set at a rate of +25 mmHg/min. and a descent time 310 set at −25 mmHg/min. In other embodiments of the therapy system 100, the triangular waveform may vary between negative pressure of 25 and 125 mmHg with a rise time 305 set at a rate of +30 mmHg/min and a descent time 310 set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

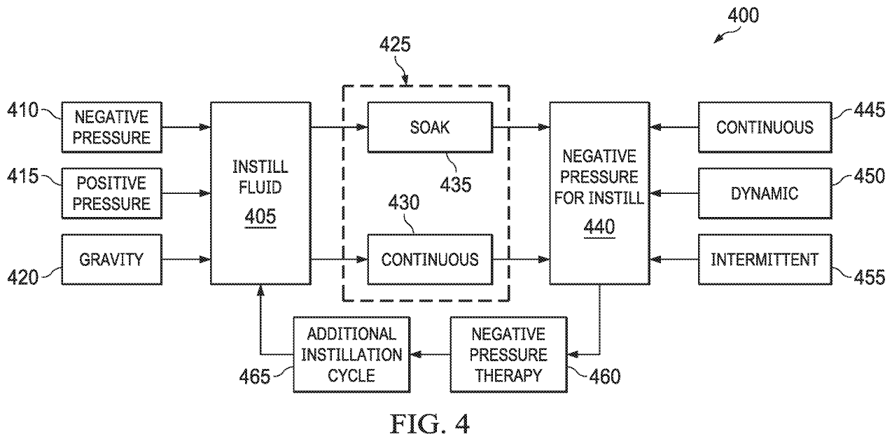

FIG. 4 is a chart illustrating details that may be associated with an example method 400 of operating the therapy system 100 to provide negative-pressure treatment and instillation treatment to the tissue interface 120. In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution, as indicated at 405. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120, as indicated at 410. In some embodiments, solution may be instilled to a tissue site by applying a positive pressure from the positive-pressure source 160 to move solution from the solution source 145 to the tissue interface 120, as indicated at 415. Additionally or alternatively, the solution source 145 may be elevated to a height sufficient to allow gravity to move solution into the tissue interface 120, as indicated at 420.

The controller 130 may also control the fluid dynamics of instillation at 425 by providing a continuous flow of solution at 430 or an intermittent flow of solution at 435. Negative pressure may be applied to provide either continuous flow or intermittent flow of solution at 440. The application of negative pressure may be implemented to provide a continuous pressure mode of operation at 445 to achieve a continuous flow rate of instillation solution through the tissue interface 120, or it may be implemented to provide a dynamic pressure mode of operation at 450 to vary the flow rate of instillation solution through the tissue interface 120. Alternatively, the application of negative pressure may be implemented to provide an intermittent mode of operation at 455 to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied at 460. The controller 130 may be utilized to select a mode of operation and the duration of the negative pressure treatment before commencing another instillation cycle at 465 by instilling more solution at 405.

In addition to negative pressure wound therapy, a dressing disclosed herein may also be used as a secondary wound dressing for treating a tissue site.

### Differential Collapse

As discussed above, the dressing 110 may comprise the tissue interface 120 and the cover 125. Additionally, the tissue interface 120 may comprise or consist essentially of one or more manifolds. When used in negative-pressure therapy, the negative pressure may provide a differential volume change within or between one or more manifolds in the tissue interface 120, for example, due to different firmness values of the one or more manifolds.

In some example embodiments, a manifold disclosed herein may be a felted manifold. Felted manifolds having different firmness values within or between manifolds may allow for varying compression or “collapse” during negative-pressure wound therapy. Therefore, in some embodiments, the tissue interface 120 may comprise or consist essentially of one or more manifolds, wherein at least one of the manifolds is a felted manifold (e.g. a felted foam), and the one or more manifolds are configured to differentially collapse during negative-pressure therapy.

Felting is a known thermoforming process that permanently compresses a material. For example, in order to create felted foam, such as felted polyurethane, the foam is heated to an optimum forming temperature during the polyurethane manufacturing process and then it is compressed. The degree of compression controls the physical properties of the felted foam. For example, felted foam has an increased effective density and felting can affect fluid-to-foam interactions. As the density increases, compressibility or collapse decreases. Therefore, manifolds, such as various foams, which have different compressibility or collapse have different firmness values. The firmness of a felted manifold, e.g. felted foam, is the felting ratio: original thickness/final thickness. In some example embodiments, a felted manifold “firmness” value or degree can range from about 1 to about 10, preferably about 1 to about 5, and more preferably from about 1 to about 3. For example, foam found in a GRANUFOAM™ dressing available from Kinetic Concepts, Inc. of San Antonio, Tex. may be felted to a density three times that of its uncompressed form. This would be referred to as firmness 3 felting. There is a general linear relationship between firmness level, density, pore size (or pores per inch) and compressibility under negative pressure. For example, foam found in a GRANUFOAM™ dressing that is felted to firmness 3 will not only show a three-fold density increase, but will only compress to about a third of its non-felted form.

In some example embodiments, the tissue interface 120 may comprise one, two or three felted manifolds, which can be used alone or in combination with one, two, three or more non-felted manifolds. Thus, the tissue interface 120 may comprise combinations of non-felted and felted manifolds. For example, in some embodiments, at least two or three of the manifolds are felted and at least one, or two, or three of the manifolds are non-felted. Each manifold may have the same or different firmness. In some embodiments, two or more manifolds may be present each having a different firmness. In additional embodiments, three or more manifolds may be present each having a different firmness.

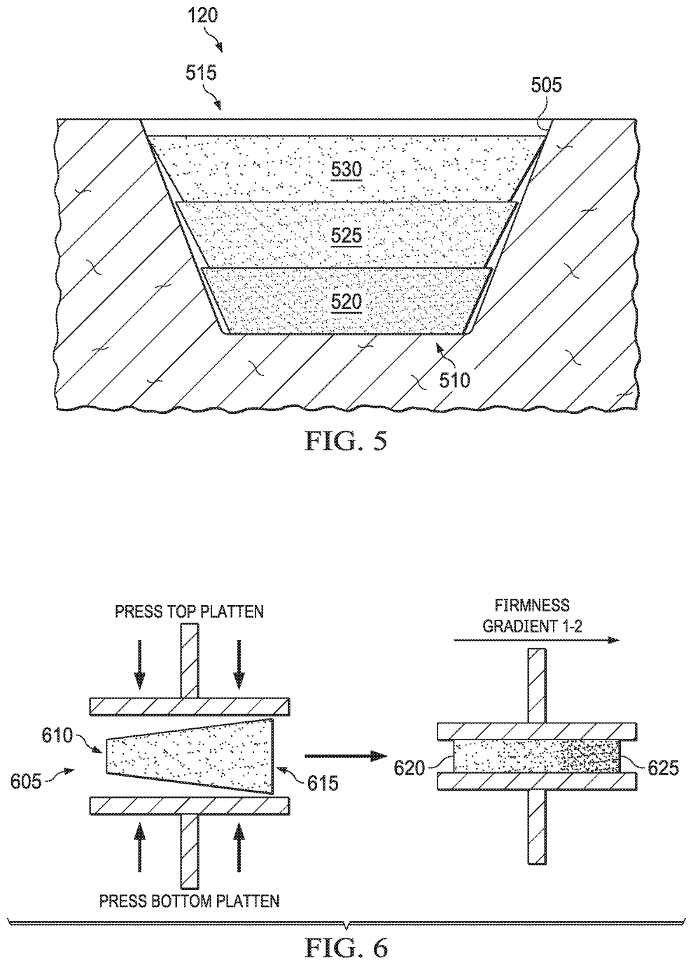

In some example embodiments, the tissue interface 120 may comprise at least two opposing surfaces, and at least one of the surfaces may be oriented or configured to face a wound bottom or bed. For example, the tissue interface 120 may comprise a first manifold having a lower firmness (i.e. high collapse) configured to be placed on a wound bottom, and a second manifold having a higher firmness (i.e. lower collapse) can be placed above the first manifold on a side opposite the wound bottom. This may encourage the wound to close from the bottom up. For example, FIG. 5 depicts the tissue interface 120 in a wound 505 having three manifolds. The first manifold 520 having a lower firmness (e.g. firmness 1) is configured to be placed at the wound bottom 510 of the wound 505. A second manifold 525 having an intermediate firmness (e.g. firmness 2) is placed over top or above the first manifold 520, and a third manifold 530 having the highest firmness and thus lowest collapse (e.g. firmness 3) is configured to be placed above the second manifold 520, near an opening 515 of the wound 505.

Additionally or alternatively, the tissue interface 120 may comprise one or more manifolds having two or more sections with different firmness, such that the manifold can have a firmness gradient. For example, one or more manifolds may be present and have one section with a lower firmness value (e.g. firmness 1 or 2) and another section with a higher firmness value (e.g. firmness 2 or 3). A firmness gradient in a manifold may be created by graded felting as shown in the example of FIG. 6. In the example of FIG. 6, a manifold 605 has a first end 610 that is less thick than a second end 615. After the manifold 605 is compressed, for example with a top and bottom platten, the manifold 605 now has a lower firmness end 620, with for example a firmness value of 1, and a higher firmness end 625, with for example a firmness value of 2. The manifold 605 can now be said to be a graded felted manifold. A graded felted manifold can be advantageous for example when an end user can cut a graded felted manifold into parts having different firmness values to use in the tissue interface 120.

Additionally or alternatively, a manifold used in the dressings disclosed herein (felted or non-felted) can have two or more partial cuts to allow further changes in compressibility. The partial cuts may not go all the way through the one or more manifold. Partial cuts can allow a manifold to collapse in on itself and to provide one or more removable parts, such as partial pillars. Any suitable cutting means can be used for creating the partial cuts. For example, hot wire, laser cutting, die cutting with limited force, or wire jet may be used. Cutting one or more manifolds to create the partial cuts can be performed before or after the polymer film discussed below is applied or contacted to a manifold, preferably before.

Additionally or alternatively, one or more of the manifolds (felted or non-felted) may be perforated. This may facilitate collapse of the one or more manifolds under pressure. Any suitable means can be used to perforate such as die cutting or slitting.

### Polymer Film

In some embodiments, the tissue interface 120 may further comprise, in addition to one or more manifolds, a polymer film coupled to the one or more manifolds.

The polymer film may comprise or consist essentially of a means for controlling or managing fluid flow. In some embodiments, the polymer film may be a fluid control layer comprising or consisting essentially of a liquid-impermeable, elastomeric material. For example, the polymer film may comprise or consist essentially of a polymer film, such as a polyurethane film. In some embodiments, the polymer film may comprise or consist essentially of the same material as the cover 125. The polymer film may also have a smooth or matte surface texture in some embodiments. A glossy or shiny finish better or equal to a grade B3 according to the SPI (Society of the Plastics Industry) standards may be particularly advantageous for some applications. In some embodiments, variations in surface height may be limited to acceptable tolerances. For example, the surface of the polymer film may have a substantially flat surface, with height variations limited to 0.2 mm over a cm.

In some embodiments, the polymer film may be hydrophobic. The hydrophobicity of the polymer film may vary, but may have a contact angle with water of at least ninety degrees in some embodiments. In some embodiments the polymer film may have a contact angle with water of no more than 150 degrees. For example, in some embodiments, the contact angle of the polymer film may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles herein represent averages of 5-9 measured values, discarding both the highest and lowest measured values. The hydrophobicity of the polymer film may be further enhanced with a hydrophobic coating of other materials, such as silicones and fluorocarbons, either as coated from a liquid, or plasma coated.

The polymer film may also be suitable for welding to other layers, including to the one or more manifolds. For example, the polymer film may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene.

The area density of the polymer film may vary according to a prescribed therapy or application. In some embodiments, an area density of less than 40 grams per square meter may be suitable, and an area density of about 20-30 grams per square meter may be particularly advantageous for some applications.

In some embodiments, for example, the polymer film may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

Additionally, the polymer film may have one or more fluid restrictions, which can be distributed uniformly or randomly across the polymer film. The fluid restrictions may be bi-directional and pressure-responsive. For example, each of the fluid restrictions generally may comprise or consist essentially of an elastic passage that is normally unstrained to substantially reduce liquid flow, and can expand or open in response to a pressure gradient. In some embodiments, the fluid restrictions may comprise or consist essentially of perforations in the polymer film. Perforations may be formed by removing material from the polymer film. For example, perforations may be formed by cutting through the polymer film, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid restrictions may be an elastomeric valve that is normally closed when unstrained to substantially prevent liquid flow, and can open in response to a pressure gradient. A fenestration in the polymer film may be a suitable valve for some applications. Fenestrations may also be formed by removing material from the polymer film, but the amount of material removed and the resulting dimensions of the fenestrations may be up to an order of magnitude less than perforations, and may not deform the edges.

For example, some embodiments of the fluid restrictions may comprise or consist essentially of one or more slits, slots or combinations of slits and slots in the polymer film. In some examples, the fluid restrictions may comprise or consist of linear slots having a length less than 4 mm and a width less than 1 mm. The length may be at least 2 mm, and the width may be at least 0.4 mm in some embodiments. A length of about 3 mm and a width of about 0.8 mm may be particularly suitable for many applications, and a tolerance of about 0.1 mm may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

### Additional Components

In some embodiments, a dressing comprising the tissue interface 120 may comprise other components in addition to the one or more manifolds and polymer film. For example, an additional component, such as an adhesive and/or an anti-microbial agent, may be interposed between one or more manifolds and a polymer film. Additionally or alternatively, the additional component, such as an adhesive and/or an anti-microbial agent, may be incorporated into one or more manifolds, or a polymer film.

One or more of the components of the dressing 110 may additionally be treated with an anti-microbial agent in some embodiments. For example, the one or more manifold may be a foam, mesh, or non-woven coated with an anti-microbial agent. In some embodiments, the one or more manifold may comprise antimicrobial elements, such as fibers coated with an anti-microbial agent. Additionally or alternatively, some embodiments of the polymer film may be a polymer coated or mixed with an anti-microbial agent. Suitable antimicrobial agents may include, for example, metallic silver, PHMB, iodine or its complexes and mixes such as povidone iodine, copper metal compounds, chlorhexidine, or some combination of these materials.

Additionally or alternatively, one or more of the components may be coated with a mixture that may include citric acid and collagen, which can reduce bio-films and infections. For example, the one or more manifolds may be a foam coated with such a mixture.

### Methods to Make

Also disclosed herein are methods of making the tissue interface 120. In some embodiments, the methods comprise felting at least one manifold, for example a foam, to a desired degree of firmness, for example 1, 2, or 3. As discussed above, felting is a well-known thermoforming process whereby material, such as foam, is permanently compressed.

In some example embodiments, one, two, three or four felted manifolds, such as a felted foam, may be configured to provide differential collapse during negative-pressure therapy. For example, two or three or four manifolds may be placed in a stacked configuration with a manifold having the lowest firmness value on one end (e.g. a wound bottom side) and a manifold having the highest firmness value on another end (e.g. a wound opening side). As shown in the example of FIG. 5, a first manifold 520 having a firmness of 1 can be placed on the wound bottom 510, then a second manifold 525 having a firmness of 2 can be placed over the first manifold 520, and a third manifold 530 having a firmness of 3 can be placed over the second manifold 525. Additionally, in some example embodiments one, two, three or more felted manifolds may be placed in a stacked configuration with one, two, three or more non-felted manifolds.

Additionally or alternatively, one, two, three or more graded felted manifolds may be placed in a stacked configuration with one, two, three or more felted manifolds and/or one, two, three, or more non-felted manifolds.

In some example embodiments, it can be advantageous to mark or indicate the degree of firmness on a manifold, for example by color coding or printing on the manifold to assist an end user to customize the tissue interface 120 for use in the dressing 110.

In further example embodiments, the methods to make the tissue interface 120 may further comprise laminating a polymer film, as described herein, to one or more manifolds. A polymer film may be laminated to one, two or three manifolds present in the tissue interface 120. In some embodiments, the methods comprise heating a surface of the one or more manifolds to provide an adhesive surface, and then coupling the polymer film to one or more manifolds present. In further embodiments, methods to make the tissue interface 120 can also include fenestrating the polymer film, preferably before laminating to the one or more manifolds.

In some example embodiments, the felting and laminating steps are done in a substantially one-step process. Alternatively, the felting and laminating steps may be performed in a two-step process, wherein the laminating is performed before or after the felting.

### Kits

Also disclosed herein are wound therapy kits comprising the tissue interface 120 described herein. A wound therapy kit may comprise multiple components which may or may not be co-packaged together. The wound therapy kits may comprise two or more manifolds having different firmness, optionally having a fenestrated polymer film laminated thereon, wherein at least one of the manifolds is felted, such as a felted foam described herein. One or more manifolds may also be a graded felted foam. The kits may further comprise one or more covers, such as a drape; and one or more dressing interfaces, such as a SENSAT.R.A.C.™ Pad available from Kinetic Concepts, Inc. of San Antonio, Tex. End users may be able to use the wound therapy kit to customize the tissue interface 120 (e.g. a wound filler) for the dressings described herein for use during negative-pressure therapy.

The systems, apparatuses, and methods described herein may provide significant advantages. For example, the different firmness values of the manifolds will allow for differential volume collapse during negative-pressure therapy, and also allow for low ingrowth and high granulation. The end user may desire to have different locations within the same wound experience a lower closure force, such as a delicate or sensitive location, or different types of wounds requiring less collapse under negative pressure.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for use with negative-pressure wound therapy comprising:
one or more manifolds, wherein at least one of the one or more manifolds is a felted manifold and the one or more manifolds are configured to differentially collapse during negative-pressure therapy; and
a polymer film having fenestrations coupled to the one or more manifolds.

2. The dressing of claim 1, comprising two or more manifolds each having a different firmness.

3. The dressing of claim 1, wherein the one or more manifolds have two or more sections with different firmness.

4. (canceled)

5. The dressing of claim 1, wherein the one or more manifolds comprise two or more manifolds in a stacked configuration.

6. The dressing of claim 1, wherein the one or more manifolds comprise at least two felted manifolds.

7. The dressing of claim 1, wherein the one or more manifolds are perforated or have one or more partial cuts.

8. (canceled)

9. (canceled)

10. (canceled)

11. (canceled)

12. The dressing of claim 1, further comprising an additional layer interposed between the one or more manifolds and the polymer film, wherein the additional layer comprises an adhesive or an anti-microbial agent or both.

13. (canceled)

14. The dressing of claim 1, wherein the polymer film is laminated to the one or more manifolds.

15. (canceled)

16. (canceled)

17. A method of making the dressing of claim 1, comprising:
felting at least one of the one or more manifolds to a desired degree of firmness;
laminating the polymer film to the one or more manifolds.

18. The method of claim 17 further comprising placing two or more manifolds in a stacked configuration.

19. The method of claim 17, further comprising fenestrating the polymer film.

20. The method of claim 17, wherein the felting comprises graded felting the one or more manifolds.

21. The method of claim 17, further comprising marking a degree of firmness on the one or more manifolds.

22. The method of claim 17, wherein the felting and the laminating are done in a one-step process.

23. (canceled)

24. The method of claim 17, further comprising perforating the one or more manifolds.

25. The method of claim 17, further comprising partially cutting the one or more manifolds to provide one or more removable parts.

26. The method of claim 17, further comprising heating a surface of the one or more manifolds to provide an adhesive surface.

27. The method of claim 17, further comprising providing an additional layer comprising an adhesive and/or anti-microbial agent interposed between the one or more manifolds and the polymer film.

28. (canceled)

29. (canceled)

30. (canceled)

31. (canceled)

32. A method of treating a tissue site with negative pressure, the method comprising:
applying the dressing of claim 1 to the tissue site;
sealing the dressing to epidermis adjacent to the tissue site;
fluidly coupling the dressing to a negative-pressure source; and
applying negative pressure from the negative-pressure source to the dressing and
promoting healing and tissue granulation.

33. The method of claim 32, wherein the negative pressure provides a differential volume change within or between the one or more manifolds.

34. (canceled)

35. (canceled)

36. (canceled)
